(19) United States
(12) Patent Application Publication
(10) Pub. No.: US 2014/0316416 A1
(43) Pub. Date: Oct. 23, 2014

(54) PATIENT-SPECIFIC GUIDANCE SYSTEM AND ACETABULAR COMPONENT WITH OFFSET STEMS FOR HIP ARTHROPLASTY

(71) Applicants: Fei Liu, Columbia, SC (US); Thomas P. Gross, Columbia, SC (US)

(72) Inventors: Fei Liu, Columbia, SC (US); Thomas P. Gross, Columbia, SC (US)

(21) Appl. No.: 13/867,738

(22) Filed: Apr. 22, 2013

(51) Int. Cl. A61B 17/17

(52) U.S. Cl. CPC A61B 17/1746; A61B 17/1703
USPC 606/87

(57) ABSTRACT

A method provide a patient-specific guidance system developed from 3D bone models, which are generated based on the patient's 2D image data. The surgical guide that is generated from this 3D data has an exterior surface that conforms to the interior surface of the acetabulum of an individual patient. Once this custom guide is placed into the patient's acetabulum, desired 3D implant position data can be transferred from the preoperative image to the acetabulum during an operation. The custom made guide allows surgeons to place guide rods into either the acetabulum itself, or into the sidewall of the pelvis (ileum or ischium) according to a preoperative plan. This guiding system is also coupled with an offset stemmed acetabular component to allow precise implantation and secure fixation of an acetabular component in a predetermined 3-D position relative to the pelvis.

### CROSS-REFERENCE TO RELATED APPLICATIONS

This application claims the benefit of U.S. Provisional Application No. 61/603,396, filed on Apr. 24, 2012.

The disclosures of the above applications are incorporated herin by reference.

### INTRODUCTION

Hip arthroplasties have been widely performed all around the world for the last few decades. Over 250,000 hip arthroplasties are performed annually in the United States. Metal-on-metal hip resurfacing arthroplasties have reemerged as promising options for young and active patients who are at a high risk for failure with standard arthroplasties. However, failures related to malposition of acetabular components are still major causes of failure with these implants. Accurately placing the acetabular component in specific safe ranges of both inclination and anteversion determined radiographically could significantly reduce failures related to adverse wear. Furthermore, the ability to place the acetabular component in an optimal 3D (three-dimensional) position is also important for hip arthroplasties utilizing other bearing surfaces. Patient specific (custom) guidance systems based on CT (computed tomography) or MRI (Magnetic resonance imaging) or other 2D (two-dimensional) data could guide a surgeon in accurately placing implants in a preoperatively planned 3D position in surgery.

The present invention involves an image based patient-specific (custom) guidance system coupled with an offset stemmed acetabular component to allow precise implantation and secure fixation of an acetabular component in a predetermined 3D position relative to the pelvis.

### SUMMARY

There has thus been outlined, rather broadly, some of the features of the invention in order that the detailed description thereof may be better understood, and in order that the present contribution to the art may be better appreciated. There are additional features of the invention that will be described hereinafter.

In this respect, before explaining the invention in detail, it is to be understood that the invention is not limited in its application to the details of construction or to the arrangements of the components set forth in the following description or illustrated in the drawings. The invention is capable of other embodiments and of being practiced and carried out in various ways. Also, it is to be understood that the phraseology and terminology employed herein are for the purpose of the description and should not be regarded as limiting.

The present invention describes a patient-specific guidance system developed from 3D bone models, which are generated based on the patient's 2D image data, such as CT or MRI images. The surgical guide that is generated from this 3D data has an exterior surface that conforms to the interior surface of the acetabulum of an individual patient. Once this custom guide is placed into the patient's acetabulum, desired 3D implant position data can be transferred from the preoperative image to the acetabulum during an operation. The custom made guide allows surgeons to place guide rods into either the acetabulum itself, or into the sidewall of the pelvis (ileum or ischium) according to a preoperative plan. One of these guide rods determines the position of the central axis of the acetabular component to orient the surgeon for preparing the acetabular bone for the acetabular component. The other guide rod determines the position and 3D orientation of the offset stem. Additional guide rods can be placed into the ileum and ischium outside of the acetabulum in order to attach jigs that can aid in accurately removing acetabular bone in preparation for the acetabular implant. The entire system allows the surgeon to first accurately remove bone for the acetabular component, then place a stem hole if desired, and finally precisely place the acetabular component according to the pre-operative surgical plan.

There are many possible versions of guidance systems that can be developed to transfer the preoperatively determined implant position information from the 3D image data to the patient's bone. We provide several representative examples, but do not wish to limit this patent only to these particular guidance systems. The first primary part of this invention is the concept of transferring implant position to the patient's bone by the method described above. The second primary part of this invention is an acetabular component with an offset stem as described above.

The above method of acetabular implant positioning can work with any standard acetabular component for hip arthroplasty but is especially effective when combined with the offset stemmed acetabular component.

Acetabular components with standard offset stems or various modular stems can be manufactured, or patient-specific implants can be produced. The guidance system, however, has to be manufactured specifically for each patient. A rapid prototyping or other suitable manufacturing method is required.

Multiple offset stems can be used if necessary. Correspondingly, the patient-specific guides can be designed to allow preparation of multiple stem positions.

### BRIEF DESCRIPTION OF THE DRAWINGS

Various other objects, features and attendant advantages of the present invention will become fully appreciated as the same becomes better understood when considered in conjunction with the accompanying drawings, in which like reference characters designate the same or similar parts throughout the several views, and wherein:

FIG. 1: is a perspective view of a component (100) with an offset stem (101) and a patient specific guidance system (200) of the present invention. Also the pelvis (300) and acetabulum (301) is shown.

FIG. 2 A: is a perspective view of a custom guide system A (200) of the present invention.

FIG. 2 B: is a perspective view of another custom guidance system C (300) of the present invention.

FIG. 3: is a perspective view of an acetabular component (100) with an offset stem (101) of the present invention.

FIG. 4 A: is a perspective view of a custom guide system A of the present invention placed into the corresponding acetabulum.

FIG. 4 B: is a perspective view of a custom guide system B of the present invention placed into the corresponding acetabulum.

FIG. 4 C: is a perspective view of a custom guide system C of the present invention placed into the corresponding acetabulum.

FIG. 4 D: is a perspective view of a custom guide system D of the present invention placed into the corresponding acetabulum.

FIG. 5: is a perspective view of an acetabular component (100) with a modular stem of the present invention.

FIG. 6: is a cross sectional view of implanting a standard acetabular component (603) into pelvis with a pre-drilled offset hole using a guiding rod (601) and a guide jig (602).

FIG. 7: is a cross sectional view of implanting a standard acetabular component (702) into pelvis through pre-drilled offset hole with use of removable jig with a stem (701).

### DETAILED DESCRIPTION OF THE INVENTION

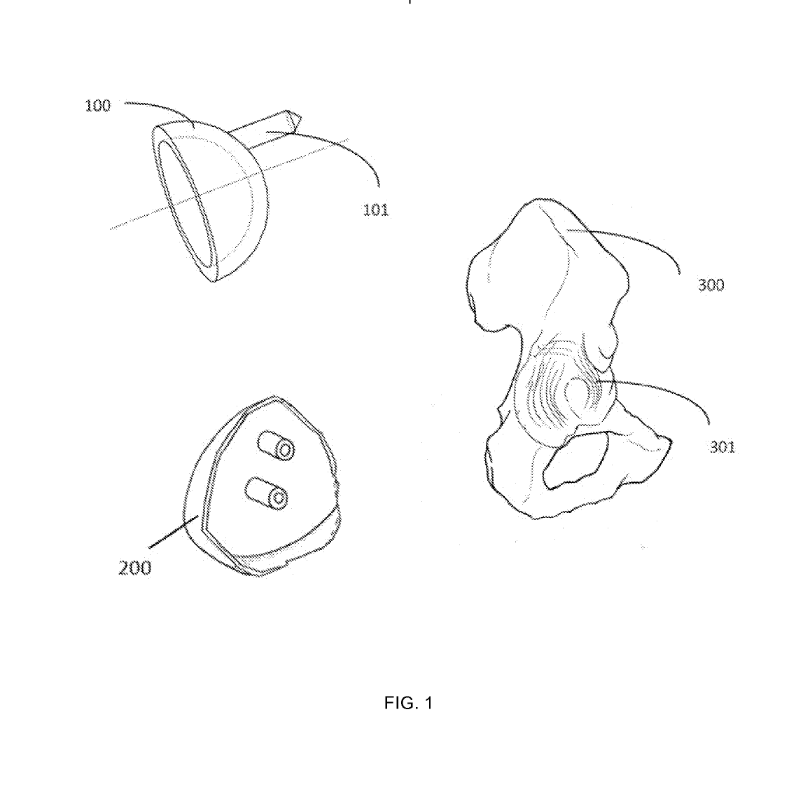

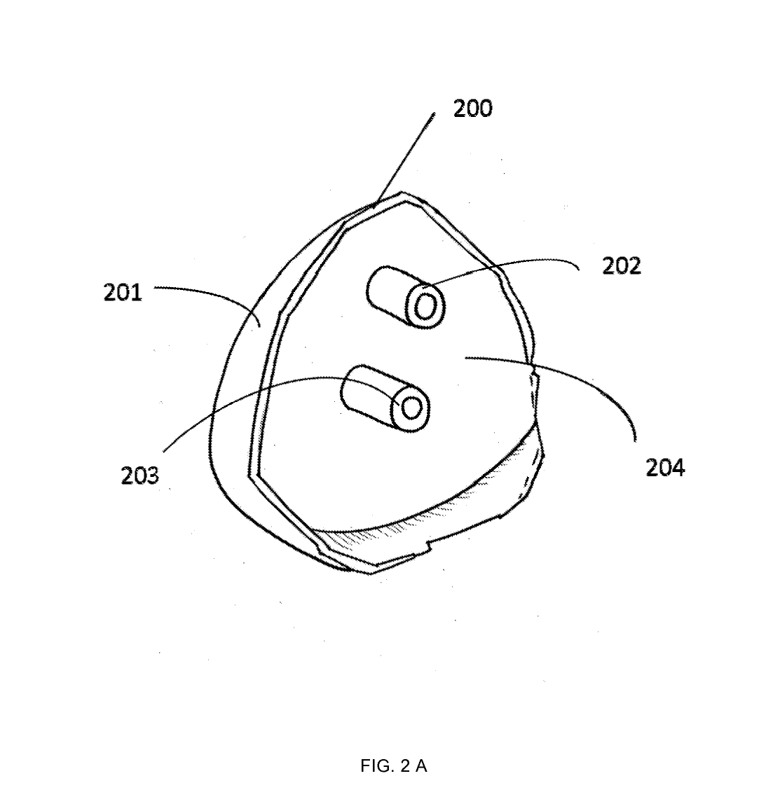

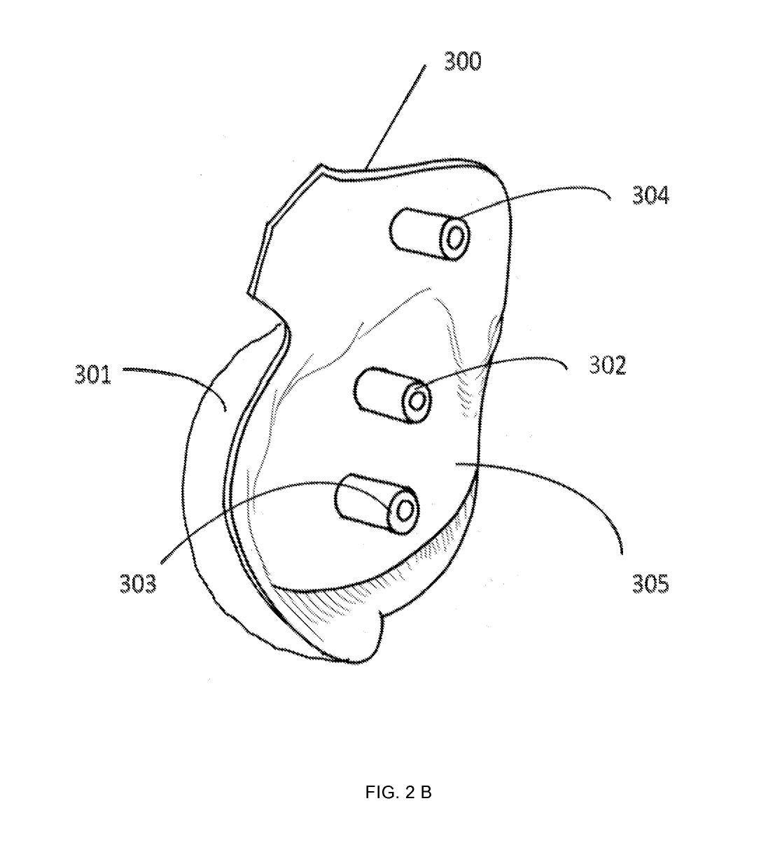

Referring to FIG. 1, the invention includes an acetabular component 100 with an offset stem 101 and a patient-specific guidance system 200 in order to match the acetabulum 301 of the pelvis 300. Referring to FIG. 2 A, the patient-specific guidance system 200 has a specific exterior surface 201, one or more drill guiding options 202 and 203 and a guide plane 204. First, a series of 2D images of the patient's pelvis and femur are acquired preoperatively. 3D CAD (Computer-aided design) models are generated from these images, which are used to accurately rebuild the pelvis and femur in the computer. Physical 3D models can be manufactured using a rapid prototyping machine and other methods. Pre-operative planning is performed based on these 3D CAD models on the computer and/or the physical models. With the help of 3D data and preoperative planning stations, the acetabular component is sized and a specific position is chosen in which to implant it. Any slightly modified standard acetabular implant can be placed with this system. Custom guides 200 are manufactured (for example with use of a rapid prototyping machine), sterilized, and be brought into the operation.

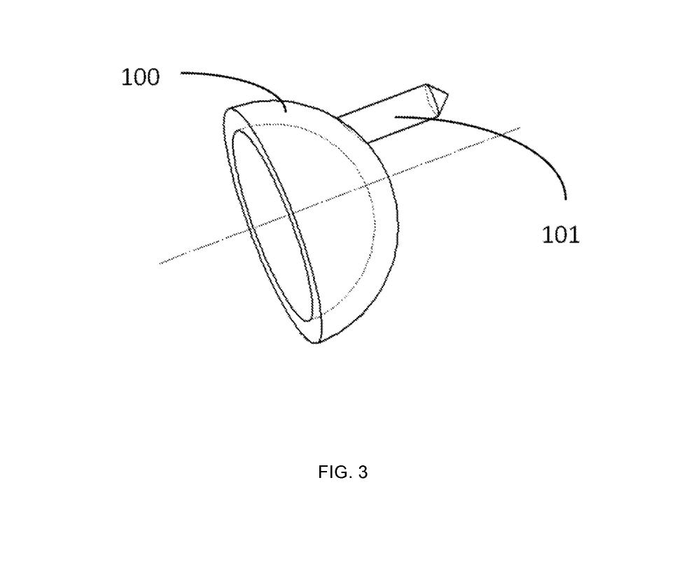

The exterior surface 201 of this patient-specific guidance system 200 matches exactly the anatomy of the inside of the acetabulum 101 of the patient's pelvis 100. Two drill holes 202 and 203 through the custom guide in FIG. 2A are predetermined based on the 3D CAD models and the previously chosen implant position. The guide hole 203 is placed in the planned position of the central axis of the hemispherical acetabular component. It determines the central axis of the hemispherical cavity that will be created by bone removal to accommodate the acetabular component. The position and orientation of this guide hole 203 are determined based on the central axis of the acetabular component to be implanted and the preoperatively selected orientation of the acetabular component and the 3D characteristics of the patient's anatomy. The guide hole may or may not be located in the center of the custom guide, but it is definitely in the position of the central axis of the future implanted acetabular component. Furthermore, the depth of acetabular preparation can be determined by using a specific guide rod with a scale or a stop. Additionally, the depth can be determined based on jigs attached to rods placed outside of the acetabular cavity. The outer “face” of the custom guide 204 determines the final resting plane of the “face” of the acetabular component. 204 also can only determine the direction of the final component orientation or other relationship between the acetabular component of the implants and the pelvis or other bones. 204 can be in any type of geometric shape. The guide hole 202 is paralleled to the guide hole 203. It determines the location and central axis of the offset stem 101 in FIG. 3. (A non-parallel arrangement could be created with the custom guide if desired for a particular situation).

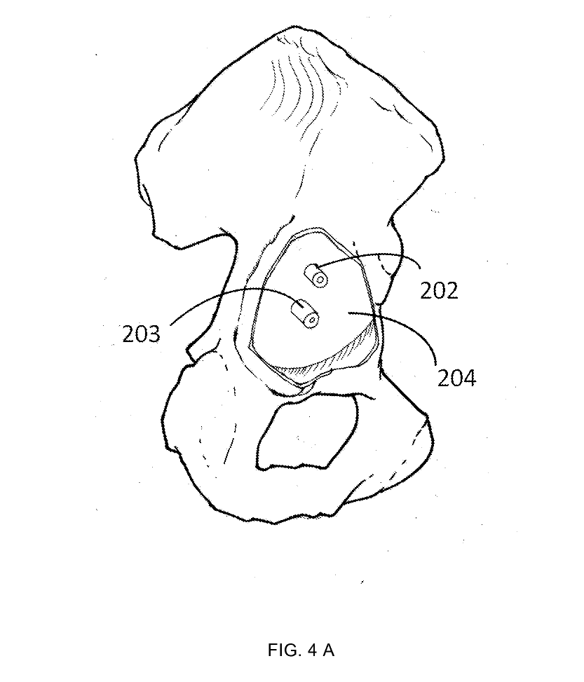

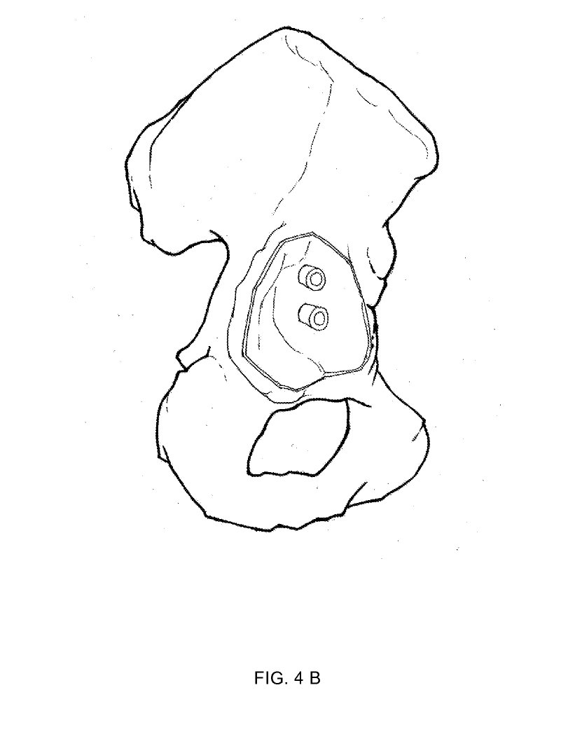

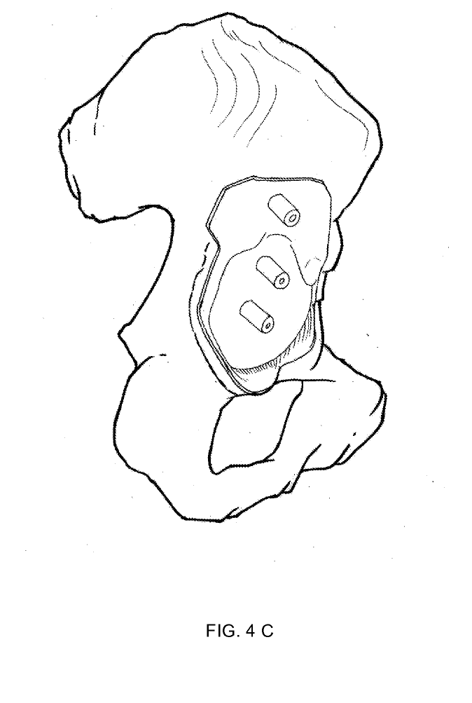

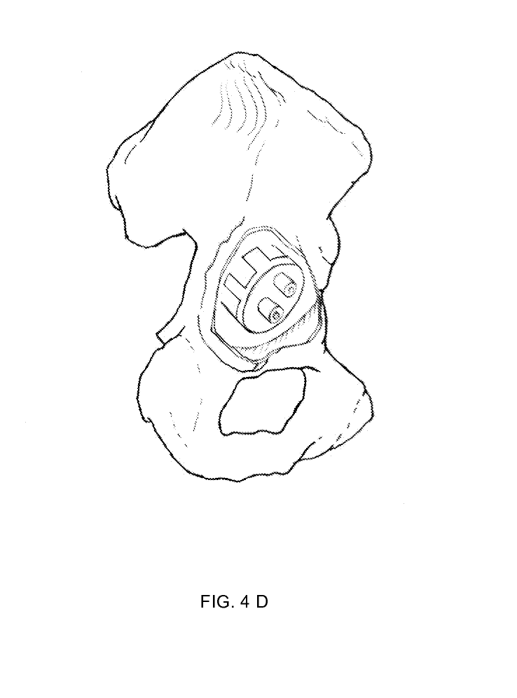

FIG. 4 A illustrates the guide system 200 inserted into the patient's native acetabulum. The number and size of the guide hole(s) (202,203) can be varied according to different requirements of the patient's anatomy or the implant. Additional holes in the guide may be required to anchor it securely to the pelvic bone. Alignment jigs and cutting tools can then be driven directly off of these guide rods to aid in accurate bone preparation and implant positioning. The jigs and machining tools may employ scales or stops to aid the surgeon in accurate preparation and implantation.

Alternatively, navigation systems or robotically driven tools could be employed in an attempt to make these processes even more accurate. A rigid frame can be fixed to the guide rods; a robotic cutting arm can then directly attach to this frame and can machine the pelvic bone. In this way, the computer can directly link the acquired 2D data from this patient's pelvis to a planned bone preparation

The figures in this patent only illustrate several different guide design possibilities in order to explain this concept; they are not intended to limit the scope of this invention. Multiple variations of guide and rod combinations could successfully transfer the preoperative 3D image data to the patient's bone in surgery.

The 3D CAD models of a patient can be created based on either a series of 2D images. The custom guidance system 200 is designed based on the patient-specific 3D models. The cartilage information will or will not be included in this custom guidance system based on whether the 2D images can provide this type of soft tissue information. For example, if these 3D CAD models are built from MRI images, the information of soft tissues such as cartilage can be included in this custom guidance system. During the operation, after the acetabulum is exposed, the custom guide can be immediately inserted into the acetabular cavity without initial preparation. However, if these 3D CAD models are built from CT images, the information of soft tissues including cartilage is not included in the custom guidance system. Thus, during the surgery, the soft tissues including cartilage should be completely removed before the custom guidance system can be inserted and be expected to fully conform to the anatomy of the acetabulum. Alternatively, an algorithm can be introduced to offset the patient's cartilage so that removal is not required

Referring to FIGS. 4 A and 4 B, the guidance system A can be a solid component with the plane 204, which corresponds to the orientation of the “face” of the final acetabular implant. It, also, can be a shell B with acceptable mechanical characteristics, without the guiding plane 204, as demonstrated in FIG. 4 B.

Referring to FIG. 4 B this guidance system C not only has two drill guide holes similar to the custom guides A and B, but also adds the drill guide system 304 on the sidewall of the ileum. Referring to FIG. 2 B, the drill guide 303 determines the center and orientation of the acetabular component. The drill guide 302 determines the center and orientation of the offset stem. The drill guide 304 allows placement of an additional guide rod into the bone outside of the acetabulum. This guide rod can visually orient the surgeon preparing the acetabulum with a reamer. Guide jigs can be attached to this guide rod to aid in accurate bone removal and implant positioning. More than one drill hole for multiple guide rods may be used. This will allow accurate implantation of any acetabular component, even those without a stem.

Referring to FIG. 3, the acetabular component 100 has an offset stem 101. The advantages of this type of component are twofold. First, correct placement is more accurate than with the method described above for the generic acetabular component. When a surgeon places the offset stemmed acetabular component into the patient's acetabulum, the 3D orientation of this component relative to the pelvis is automatically determined when the acetabular component is seated into the prepared cavity while the offset stem is simultaneously being inserted into the pre-drilled hole. The 3D orientation including acetabular inclination and anteversion angles and depth of implantation will then match exactly the pre-operatively selected plan. Also, initial fixation of the acetabular component will be much more secure. Once this implant is placed, the risk of shifting during the remainder of the procedure and even postoperatively will be minimized. The implant only has an opportunity to shift acutely if a fracture in the surrounding bone occurs or chronically if fixation of the implant itself fails. The stem can be treated with a bone ingrowth fixation surface, but usually we would intend the stem to be smooth for easy insertion and for initial fixation only (currently screws are commonly used to initially fix acetabular components, we would expect the stem to serve a similar function). The stem would aid in holding the acetabular shell rigidly until bone ingrowth into the shell occurred. Thereafter the stem would no longer serve a function except possibly for purposes of monitoring the implant. If the stem broke or shifted in the bone, it would be a good indication that implant fixation has failed (similar to screws currently) and revision is required. Long-term fixation would typically be provided by bone ingrowth into a suitable surface on the shell of the acetabular component, or less commonly with cement. Numerous options are available and in common use.

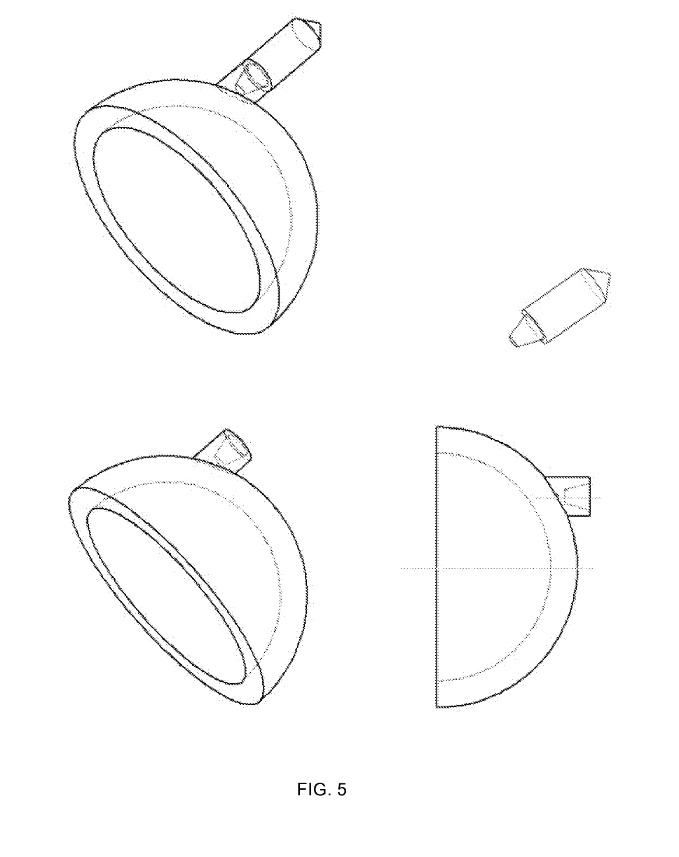

The stem could be manufactured as a single piece with the acetabular shell. The acetabular shell could provide options for fixing various liner bearing types within it as is commonly done with current acetabular systems. A metal bearing surface could be directly machined into the shell or another bearing surface (for example polyethelene or ceramic) could be fixed to the shell in advance. Basically, a stem will work as we have described it, when attached to any available acetabular system. Additionally we envision the possibility of this stem being modular and available in several sizes. The surgeon would assemble it into a standardized position on the acetabular shell with a morse-cone taper, screws or other methods. FIG. 5 demonstrates one sample of a modular offset component.

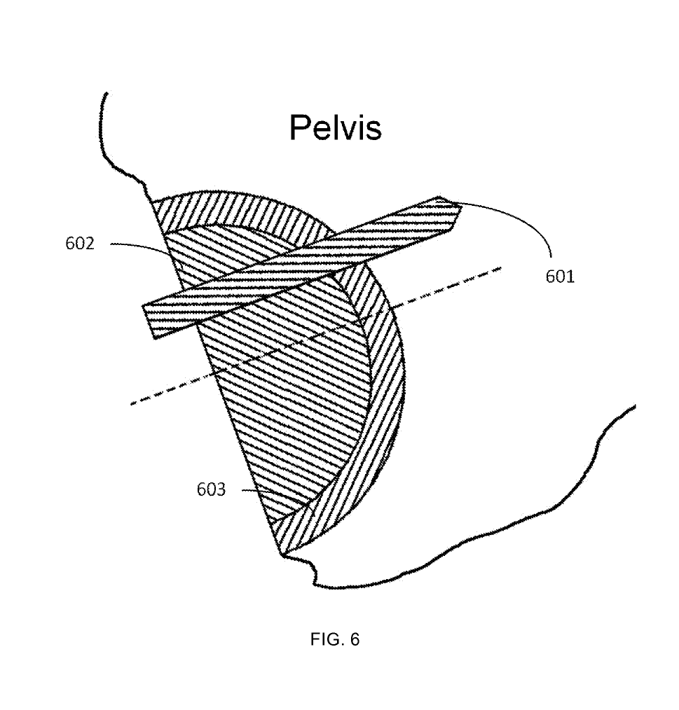

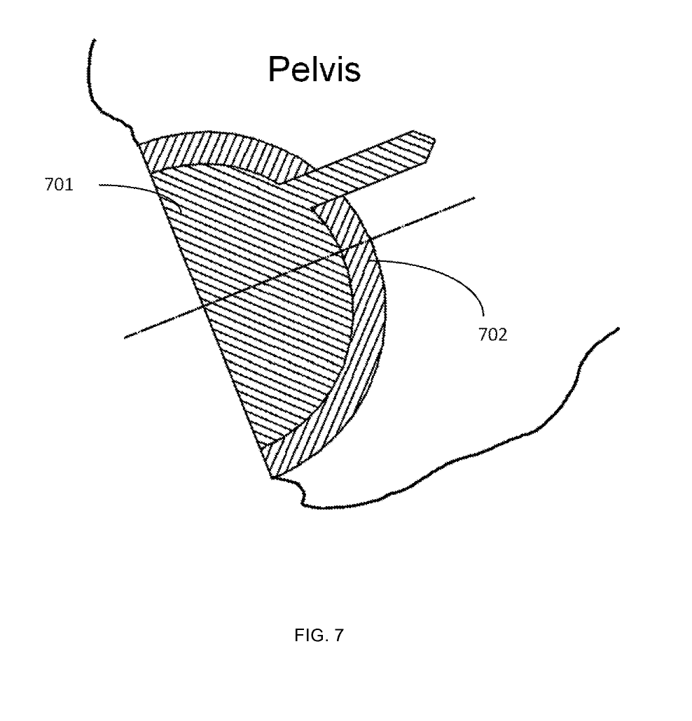

Furthermore, there are several options for a temporary guide rod/stem that could be used with an acetabular component and then removed through a hole in the component after the implant is seated. A modified component is required with an appropriate hole of the correct size and orientation. The acetabular cavity and the guide hole prepared from the original custom guide determine the desired implant position. FIG. 6 shows an example of a temporary guide rod (601) that can be used with a standardized jig (602) to guide the implant into position. The jig is locked into the implant and the unit is slid over the guide rod into position. The rod is then removed through the hole in the implant (603). The jig (602) is unlocked from the implant (603) and is also removed. FIG. 7 shows an example where the temporary guide stem and jig are combined as one piece (701). This temporary stemmed jig can be rigidly fixed to the implant (702) with a locking mechanism. After component implantation, the jig is unlocked from the implant and it is removed. Bone graft or cement can be used to fill the guide hole after implanting the standard component. If desired, screws can then be implanted in the standard fashion.

The acetabular component can be standardized and manufactured in multiple specific sizes with varying stem lengths, positions, and thicknesses. Or certain modular stems can be available to attach to standardized positions on the acetabular shell. Alternatively, custom designed acetabular components could be used.

What has been described and illustrated herein is a preferred embodiment of the invention along with some of its variations. The terms, descriptions and figures used herein are set forth by way of illustration only and are not meant as limitations. Those skilled in the art will recognize that many variations are possible within the spirit and scope of the invention in which all terms are meant in their broadest, reasonable sense unless otherwise indicated. Any headings utilized within the description are for convenience only and have no legal or limiting effect.

The primary claims of this patent are twofold:

1. A guidance system that allows transfer of a preoperatively determined implant position to the patients acetabular bone using:
A preoperative 3D bone CAD models based on image such as CT or MRI.
A custom guide created from this 3D image that fits into the native acetabulum.
One or more guide rods that are placed into the bone around the patient's acetabulum (both inside and outside of the actual acetabulum) in a predetermined position using the custom guide.
After the guide is removed, these rods precisely guide bone removal from the acetabulum using a set of jigs and cutting tools.
These guide rods facilitate precise implant positioning into the prepared acetabulum. Positioning can be with jigs based on the above guide rods, or using an offset stem described below if a stem hole is first prepared based on the guide rod position.
The custom guide can provide additional implant positioning data by having the outside “face” of the custom guide manufactured to represent the final resting plane of the planned acetabular component.

2. An acetabular component with a permanent or removable offset stem that can be used to precisely position and possibly fix the implant into the desired orientation in this hemispherically prepared acetabular bone. We claim any stem that is longer than 1 cm, parallel to the central axis of the acetabular component, but offset so that the center of the stem is not exactly in the central axis of the implant. We claim 3 possibilities for this stem:
A one-piece stem that is manufactured as part of the implant.
A modular stem that is intended as a permanent implant and is securely fixed to the acetabular component prior to component insertion.
A modular guidance stem/rod that is either temporarily fixed to the implant, but then removed after the implant is accurately implanted or that simply allows an implant to glide over it in a controlled fashion during implantation prior to being removed.
More than one stem could be used.
